(19) United States
(12) Patent Application Publication
(10) Pub. No.: US 2023/0277587 A1
(43) Pub. Date: Sep. 7, 2023

(54) FOOD SUPPLEMENT FOR ORAL ADMINISTRATION

(71) Applicant: DERMACISEN, S.A., Ciudad de Panama (PA)

(72) Inventor: Jose Luis Cisneros Vela, Barcelona (ES)

(21) Appl. No.: 17/685,666

(22) Filed: Mar. 3, 2022

(51) Int. Cl. A61K 33/42; A61K 36/87; A61K 31/375; A61K 31/122; A61K 31/7048; A61K 31/355; A61K 31/728; A61K 31/01; A61K 31/714; A61K 33/04; A61K 9/48; A61K 47/38; A61K 31/519; A23L 33/105; A23L 33/15; A23L 33/16; A23L 29/262

(52) U.S. Cl. CPC A61K 33/42; A61K 36/87; A61K 31/375; A61K 31/122; A61K 31/7048; A61K 31/355; A61K 31/728; A61K 31/01; A61K 31/714; A61K 33/04; A61K 9/4816; A61K 47/38; A61K 31/519; A23L 33/105; A23L 33/15; A23L 33/16; A23L 29/262; A23V 2002/00

(57) ABSTRACT

The present invention relates to a food supplement for oral administration, intended for human consumption, comprising capsules containing the components of the food supplement per se. Said supplement comprises, with respect to the total weight of the food supplement: between 65% and 75% of active ingredients consisting of antioxidants, vitamins, and iron; between 8% and 15% of thickening additives, anti-caking agents, and bulking agents, and between 12% and 20% of a cover forming the capsules. Said food supplement is applicable in beneficial effects in skin care, specifically in the reduction of dryness and wrinkles, the improvement of muscle tone, possible atrophies of the urogenital system, and the endogenous stimulation of defenses.

### FIELD OF THE INVENTION

The present invention relates to a food supplement for oral administration, intended for human consumption, and the main purpose of which is to prevent skin deterioration and dryness and delay skin aging.

### BACKGROUND OF THE INVENTION

In the 21^{st} century, wellness medicine and aesthetic medicine have come together in dermatology supported by the development of all types of body care products, mostly creams for topical use, known colloquially as anti-wrinkle or “anti-aging” creams.

Although these creams or products for topical use provide a skin hydrating effect and can contribute to delaying or minimizing wrinkles, they have a very limited effect because the application thereof is limited to specific areas, and mainly to the face and neck, due to their high cost.

Different hair and skin care products in the form of tablets or liquids for oral consumption are also known, the composition of which mainly includes a vitamin or a specific active ingredient to achieve a given effect.

The problem being considered is the development of a food supplement for oral administration, intended for human consumption and providing different beneficial effects for body maintenance, and mainly skin maintenance, by delaying aging.

The applicant of the invention is unaware of the existence in the state of the art of food supplements which anticipate the composition of the food supplement of the present invention.

### DESCRIPTION OF THE INVENTION

The food supplement for oral administration, intended for human consumption, object of the invention, includes in its composition a series of products and active ingredients which act together and in a synergistic manner on different aspects of the body, providing beneficial effects in skin care, specifically in the reduction of skin dryness which contributes to the reduction of wrinkles, and in other aspects such as: the improvement of muscle tone, the improvement of possible atrophies of the urogenital system, or the endogenous stimulation of defenses.

This food supplement for oral administration, with a presentation in the form of capsules, comprises a series of ingredients with different properties and functions, and with the proportions by weight with respect to the total weight of the food supplement indicated below:
between 65% and 75% of active ingredients consisting of antioxidants, vitamins, and iron,
between 8% and 15% of thickening additives, anti-caking agents, and bulking agents.
between 12% and 20% of a cover of the capsule.

According to the invention, the active ingredients of the food supplement comprise, with respect to the total weight of the food supplement: from 15% to 20% of dry grape seed extract, from 10% to 15% of ascorbic acid (vitamin C), and from 22% to 27% of ferric pyrophosphate, in combination with at least one of the following components: coenzyme Q10, rutin, DL-alpha-tocopheryl acetate (vitamin E), hyaluronic acid, Polygonum cuspidatum dry extract (resveratrol), lycopene, cyanocobalamin (vitamin B12), folic acid (vitamin B9), and sodium selenite.

The mentioned active ingredients have specific properties with different treatment functions, specifically:
grape seed extract is for improving circulation and cholesterol;
ascorbic acid (vitamin C) is an antioxidant which improves iron absorption from plant-based foods and supports the immune system;
ferric pyrophosphate is beneficial in the treatment of iron loss or deficiency;
coenzyme Q10 is an antioxidant used by body cells for growth and maintenance;
rutin performs a significant function as a circulatory system protective agent, antioxidant, and immune system reinforcement;
DL-alpha-tocopheryl acetate (50% vitamin E) is a liposoluble vitamin with high antioxidant power capable of neutralizing free radicals;
hyaluronic acid is capable of attracting and retaining water, and this causes tissues to remain in good conditions, minimizing or delaying wrinkle formation,
Polygonum cuspidatum dry extract (90% resveratrol) exhibits a very positive action as an antioxidant,
lycopene exhibits very potent antioxidant properties which contribute to evening out marks caused on the skin by the sun or by age and combats aging.
cyanocobalamin (vitamin B12) is essential for cell growth and reproduction;
folic acid (vitamin B9) performs functions relating to the nervous system and the suitable functioning of the cardiovascular system, and
sodium selenite is used as a selenium source, one of the main functions of which is to protect cells from oxidative damage and keep hair in good condition.

The combination of the active ingredients produces a synergy of or an increase in the beneficial action of the active ingredients in blood circulation, vitamin intake, skin hydration with subsequent wrinkle reduction or treatment, immune system reinforcement, and delayed aging due to the effect of the antioxidants.

As mentioned, the tablets of this food supplement include, in addition to the active ingredients, a series of thickening additives, anti-caking agents, and bulking agents.

Advantageously and by way of example, bulking agent comprises microcrystalline cellulose (E460i) at a proportion comprised between 8% and 12% with respect to the total weight of the food supplement; the product thickener comprises croscarmellose sodium at a proportion comprised between 0.5% and 2% with respect to the total weight of the food supplement, and the anti-caking product, with a proportion comprised between 0.5% and 2% with respect to the total weight of the food supplement, comprises at least one of the following components: silicon dioxide and vegetable magnesium stearate.

The cover of the capsule per se is hydroxypropyl methylcellulose.

### Detailed Description of Embodiments of the Invention

In a specific embodiment, the food supplement for oral administration is made up of capsules weighing 800 mg, of which: 677 mg correspond to the active ingredients and additives included inside the capsule and the remaining 123 mg correspond to the capsule or external covering.

In this specific example, the food supplement comprises the following composition of: active ingredients, additives, and outer cover, with percentages by weight indicated with respect to the total weight of the food supplement:

| | | % by | |
|---|---|---|---|
| Composition | mg | weight | Type |
| Grape seed extract | 150.000 | 18.750% | Active ingredient |
| Ascorbic acid (vitamin C) | 100.000 | 12.500% | Active ingredient |
| Ferric pyrophosphate | 198.582 | 24.823% | Active ingredient |
| Coenzyme Q10 | 30.000 | 3.750% | Active ingredient |
| Rutin | 26.315 | 3.289% | Active ingredient |
| DL-alpha-tocopheryl acetate | 24.000 | 3.000% | Active ingredient |
| (50% vitamin E) | | | |
| Hyaluronic acid | 21.052 | 2.632% | Active ingredient |
| Polygonum cuspidatum dry | 8.526 | 1.066% | Active ingredient |
| extract | | | |
| Lycopene | 14.000 | 1.750% | Active ingredient |
| Cyanocobalamin (vitamin B12) | 2.500 | 0.313% | Active ingredient |
| Folic acid (vitamin B9) | 0.200 | 0.025% | Active ingredient |
| Sodium selenite | 0.127 | 0.016% | Active ingredient |
| Microcrystalline cellulose | 83.698 | 10.462% | Bulking agent |
| (E460i) | | | |
| Vegetable magnesium stearate | 6.000 | 0.750% | Anti-caking |
| Silicon dioxide | 4.000 | 0.500% | Anti-caking |
| Croscarmellose sodium | 8.000 | 1.000% | Thickener |
| Hydroxypropyl methylcellulose | 123.000 | 15.375% | Cover |
| TOTAL | 800.000 | 100.000% | |

Having sufficiently described the nature of the present invention, as well as a preferred embodiment thereof, it is hereby stated for all pertinent purposes that the materials, shape, size, and arrangement of the described elements can be modified provided that said modification does not entail an alteration of the essential features of the invention that are claimed below.

1. A food supplement for oral administration, intended for human consumption, comprising capsules containing the components of the food supplement per se, characterized in that it comprises, with respect to the total weight of the food supplement:
between 65% and 75% of active ingredients consisting of antioxidants, vitamins, and iron,
between 8% and 15% of thickening additives, anti-caking agents, and bulking agents.
between 12% and 20% of a cover forming the capsules.

2. The food supplement according to claim 1, characterized in that the active ingredients comprise, with respect to the total weight of the food supplement: from 15% to 20% of dry grape seed extract, from 10% to 15% of ascorbic acid (vitamin C), and from 22% to 27% of ferric pyrophosphate, in combination with at least one of the following components: coenzyme Q10, rutin, DL-alpha-tocopheryl acetate (Vitamin E), hyaluronic acid, Polygonum cuspidatum dry extract (resveratrol), lycopene, cyanocobalamin (vitamin B12), folic acid (vitamin B9), and sodium selenite.

3. The food supplement according to claim 1, characterized in that the bulking agent comprises microcrystalline cellulose (E460i) at a proportion comprised between 8% and 12% of the total weight of the food supplement.

4. The food supplement according to claim 1, characterized in that the thickener comprises croscarmellose sodium at a proportion comprised between 0.5% and 2% with respect to the total weight of the food supplement.

5. The food supplement according to claim 1, characterized in that the anti-caking agent, at a proportion comprised between 0.5% and 2% with respect to the total weight of the food supplement, comprises at least one of the following components: silicon dioxide and vegetable magnesium stearate.

6. The food supplement according to claim 1, characterized in that the cover forming the capsule is hydroxypropyl methylcellulose.
